(19) United States
(12) Patent Application Publication
(10) Pub. No.: US 2023/0152265 A1
(43) Pub. Date: May 18, 2023

(54) DEVICE, METHOD AND SYSTEM FOR DETECTING NICOTINE

(71) Applicant: ROYAL MELBOURNE INSTITUTE OF TECHNOLOGY, Melbourne (AU)

(72) Inventors: Ataur RAHMAN, Melbourne (AU); Sumeet WALIA, Melbourne (AU); Madhu BHASKARAN, Melbourne (AU); Sharath SRIRAM, Melbourne (AU)

(73) Assignee: ROYAL MELBOURNE INSTITUTE OF TECHNOLOGY, Melbourne (AU)

(21) Appl. No.: 18/054,752

(22) Filed: Nov. 11, 2022

(30) Foreign Application Priority Data
Nov. 12, 2021 (AU) 2021903631

(51) Int. Cl. G01N 27/12; G01N 27/04; G08B 17/10

(52) U.S. Cl. CPC G01N 27/125; G01N 27/045; G08B 17/10

(57) ABSTRACT

This application discloses a device for detecting nicotine. The device includes a nicotine sensor comprising vanadium oxide (VO₂), a processor and circuitry configured to detect a change in the electrical resistance of the nicotine sensor. The processor is configured to receive, from the circuitry, a signal representative of the detected change in the electrical resistance of the nicotine sensor and output data based on the detected change in electrical resistance of the nicotine sensor. A system and method for detecting nicotine are also disclosed.

### TECHNICAL FIELD

The present disclosure relates to a device, method and system for detecting nicotine.

### BACKGROUND

Use of tobacco products, including both conventional cigarettes and electronic cigarettes (e-cigs, also known as vape or vaping), increases the risk of cardiovascular and respiratory disorder. Although electronic cigarettes were originally developed to reduce the toxic effects of smoke inhalation and assist smokers to transition to a more healthy alternative, they have become popular among people who have never smoked before, especially teenagers. Further, e-cigarettes have been found to negatively affect multiple organs including the respiratory system by altering airflow through the conducting airways, increasing oxidative stress, interfering with lung development and impairing host defence against bacterial and viral pathogens.

The risk to health extends not just to the primary smokers who directly inhale the smoke or vape gases, but also to people in the immediate vicinity of the primary smokers who are exposed to second hand smoke or vape gases and to people who later enter the area and may be exposed to third hand smoke, which is the chemical residue left by smoke and gases which linger on hair, clothing, furniture, dust and surfaces long after the original smoking has stopped.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

A first aspect of the present disclosure provides a device for detecting nicotine comprising: a nicotine sensor comprising vanadium oxide (VO₂), the nicotine sensor having an electrical resistance which is sensitive to presence of nicotine; circuitry configured to detect a change in the electrical resistance of the nicotine sensor; and a processor configured to receive, from the circuitry, a signal representative of the detected change in the electrical resistance of the nicotine sensor and configured to output data based on the detected change in electrical resistance of the nicotine sensor.

A second aspect of the present disclosure provides a method of detecting gaseous nicotine, the method comprising: exposing a vanadium oxide (VO₂) component of a nicotine sensor to gaseous nicotine; detecting a change in an electrical resistance of the VO₂ component of the nicotine sensor; and generating data based on the detected change in electrical resistance of the VO₂ component of the nicotine sensor.

A third aspect of the present disclosure provides a system for detecting nicotine comprising a first device and a second device. The first device includes a nicotine sensor comprising vanadium oxide (VO₂), a first processor, circuitry configured to monitor an electrical property of the nicotine sensor and send a signal based on the monitored electrical property to the first processor, and the first processor is configured to control a first wireless communication module to send data based on the signal to the second device. The second device comprises a second wireless communication module to wirelessly receive the data from the first device and a second processor to determine a concentration of nicotine at the nicotine sensor of the first device based on the data received from the first device.

Further aspects and features of the present disclosure are provided in the following description and the appended claims.

### BRIEF DESCRIPTION OF DRAWINGS

Examples of the present disclosure will now be described, by way of non-limiting example only, with reference to the accompanying drawings, in which:

FIG. 1 is a schematic drawing showing a device for detecting nicotine according to one example;

FIG. 2A is a graph showing how the electrical resistance of VO₂ decreases in the presence of nicotine according to one example;

FIG. 2B is a graph showing the sensitivity of VO₂ to nicotine as compared to other analytic molecules according to one example;

FIG. 3 is a schematic drawing showing a device for detecting nicotine according to another example;

FIGS. 4A and 4B show two example structures for a nicotine sensor;

FIGS. 5A to 5C show example electrode patterns for a nicotine sensor;

FIG. 6A is a circuit diagram of a nicotine detecting device and an external device wirelessly coupled to the nicotine detecting device according to another example;

FIG. 6B is an exploded diagram showing an example structure of the nicotine detecting device of FIG. 6A;

FIG. 6C is a top down diagram showing an example structure of the nicotine detecting device of FIG. 6A;

FIG. 6D is a diagram showing nicotine detecting device of FIG. 6A when worn by a person;

FIG. 7 is a block diagram illustrating a method of detecting nicotine according to one example;

FIG. 8 is a graph showing sensitivity of a nicotine detecting device to different concentrations of nicotine according to one example;

FIG. 9A shows the response of a nicotine detecting device to repeated exposures to vape according to one example;

FIG. 9B shows the response of a nicotine detecting device to repeated exposures to cigarette smoke according to one example;

FIG. 9C shows variation in response of a nicotine detecting device with changes in temperature according to one example; and

FIG. 10 shows a system for detecting nicotine comprising a first device and a second device.

### DESCRIPTION OF EMBODIMENTS

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps. The terms “includes” means includes but not limited to, the term “including” means including but not limited to. The term “based on” means based at least in part on. The term “number” means any natural number equal to or greater than one. The terms “a” and “an” are intended to denote at least one of a particular element.

As used herein the term “smoke” refers to the smoke produced by smoking a cigarette, while the terms “vape” and “vapour” refer to the gases produced by smoking an e-cigarette (also known as “vaping”). There is a demand for sensors which are capable of detecting the presence of cigarette smoke or vapours. Such sensors could be used in schools, hospitals or public places were smoking is prohibited, used in research to detect ambient levels of cigarette or vape gases, or worn by individuals to monitor whether or not they are smoking or vaping. However, traditional smoke detectors are not be able to detect the gaseous products of vaping and may be set off by other gases, or combustion products, which are unrelated to smoking.

One approach is to design a sensor which is capable of detecting nicotine, as nicotine is present in both conventional cigarette smoke and e-cigarette vapours. Not only is nicotine a marker of smoking and vaping, it is also detrimental to health because of its carcinogenic potential and therefore a substance of interest. However, it is difficult to design a sensor which is sensitive to nicotine, but not triggered by other gases which may be present in the environment. For example, in addition to nicotine, vaping may produce hundreds of different gases, not all of which are exclusive to smoking or vaping.

One approach is to detect nicotine in liquid form in the sweat of a smoker, which may be collected via a sweat band. However, this approach requires an extended period of exercise to produce the sweat and so cannot detect smoking or vaping in real time. Furthermore, this approach is not capable of detecting second or third hand smoke. Another approach is to use a gasochromic sensor which detects nicotine in the presence of sunlight by changing the colour of metal organic nanotubes. However, this approach is not very versatile as it requires the presence of sunlight and is difficult to gather and process the data. Measurement of ambient nicotine using gas chromatography and mass spectrometry is accurate, but impractical outside of a laboratory and unsuitable for continuous monitoring or detecting individual smoking/vaping events, as these techniques require long sampling periods lasting days or even weeks.

The inventors have found that vanadium oxide (VO₂) has electrical properties which are sensitive to the presence of nicotine. For example, through experimentation, the inventors have found that the presence of nicotine, in concentrations produced by smoking or vaping, reduces the electrical resistance of VO₂ at room temperature. Based upon this research, the inventors have designed various sensors, systems, devices and methods for detecting nicotine.

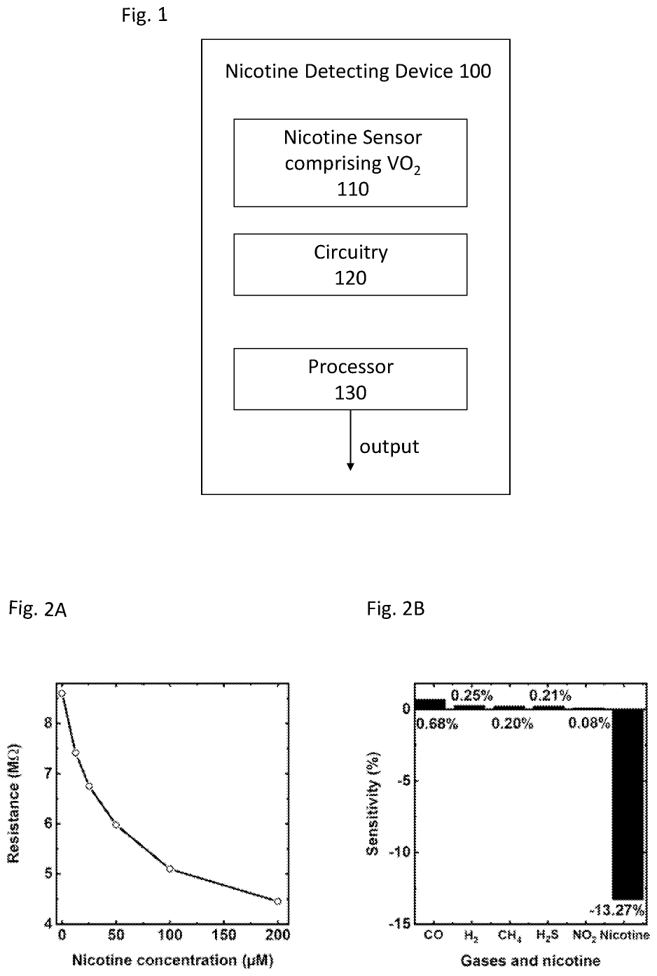

FIG. 1 shows one example of a device 100 for detecting nicotine. The device comprises a nicotine sensor 110 comprising vanadium oxide (VO₂), circuitry 120 and a processor 130. The nicotine sensor 110 has an electrical resistance which is sensitive to the presence of nicotine. The circuitry 120 is configured to detect a change in the electrical resistance of the nicotine sensor. The processor 130 is configured to receive, from the circuitry 120, a signal representative of the detected change in the electrical resistance of the nicotine sensor and to output data based on the detected change in electrical resistance of the nicotine sensor.

The circuitry 120 may be any electrical circuitry configured for detecting an electrical property, such as resistance, of the nicotine sensor. In some examples the electrical circuitry comprises a number of resistors. The processor 130 may for example be a microprocessor, a central processing unit, a logic chip such as an application specific integrated chip (ASIC) or field programmable gate array (FPGA) or other processing device etc. In some examples the processor may be a near field communications (NFC) chip.

FIG. 2A illustrates how, based on the inventors' research, the electrical resistance of VO₂ decreases in the presence of increasing nicotine concentration. This property is thought to be due to nicotine adsorbing on the VO₂ surface and acting as a charge injector. Furthermore, the VO₂ is sensitive to both liquid and gaseous forms of nicotine and highly selective for nicotine as it does not have a similar response to other tested gases. FIG. 2B shows the sensitivity of VO₂ to nicotine as compared to other analytic molecules, namely prominent gases such as CO, H₂, CH₂, H₂S and NO₂. The sensitivity, S, of the VO₂ surface to nicotine is calculated according to the following equation:

S=(Ra−Rg)/Ra×100 Equation 1

where Ra is the electrical resistance of a sensor comprising a VO₂ surface exposed to air and Rg is the electrical resistance upon application of the analytic molecules on the sensor surface. As can be seen from FIG. 2B, VO₂ shows minimal response to various other prominent gases, whereas nicotine shows strong response with sensitivity 10-100 times higher.

The circuitry 120 of the device 100 detects changes in the electrical properties of the nicotine sensor 110, such as changes in the electrical resistance. The circuitry may input signals indicative of electrical properties of the nicotine sensor, or indicative of changes in the electrical properties, to the processor 130. In this way the device 100 utilises the nicotine sensitive electrical properties of VO₂ to generate output data which may be used to determine the presence of nicotine. The output data may take any appropriate form and will depend on the system design and end user requirements. For example, in some implementations the processor 130 may output electrical values, such as voltage, current or resistance measurements, while in other examples the processor 130 may carry out further processing and output data such as calculated nicotine concentrations or other parameters related to nicotine. In still other examples, the output data may be an alert indicating the presence of nicotine, for instance if the response of the nicotine sensor 110 meets predetermined criteria.

The device 100, as described above, may detect changes in nicotine concentration in real time by detecting changes in electrical resistance of the nicotine sensor 110 in real time. The device 100 may thus be used to detect single instances of smoking in real time, unlike sensors which require long exposure times. The device may for instance be installed in a room, for example in a school or hospital, and used as an alarm, or provided as a wearable device to be worn by a person in order to monitor whether the person is smoking, provide information on the level of nicotine, or monitor exposure to second hand or third hand cigarette smoke or vapours.

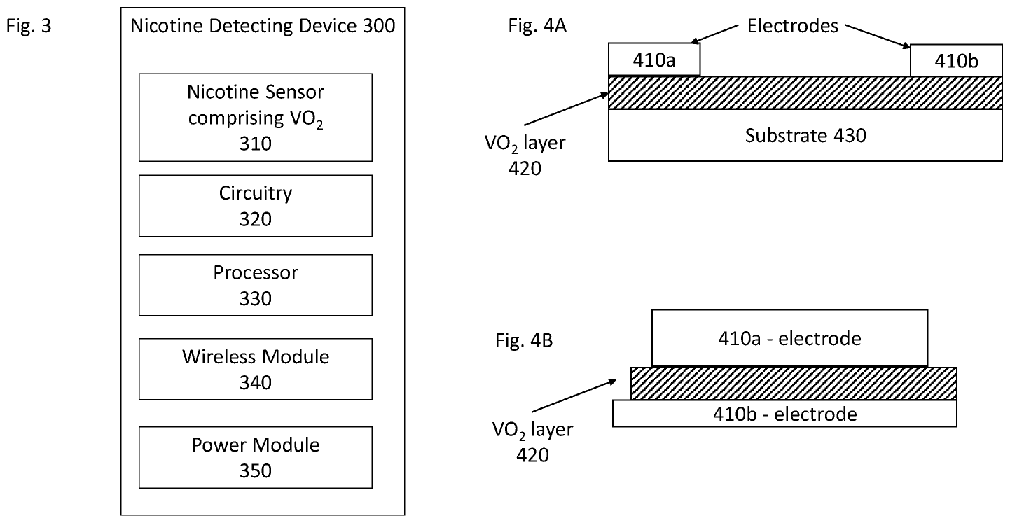

FIG. 3 shows a further example of a device 300 for detecting nicotine. The device 300 comprises a nicotine sensor 310, circuitry 320 and a processor 330 which may be the same as the nicotine sensor 110, circuitry 120 and a processor 130 described above with reference to FIG. 1.

The device 300 further comprises a wireless module 340 for communicating the output data to an external device. The wireless module 340 may for instance be in communication with the processor 330 and may be configured to send wireless communications to and/or receive wireless communications from an external device, such as but not limited to a smart phone, smart watch, computer server, cloud based service etc. In some examples, the wireless module is configured to communicate with a smart phone or smart watch, which may be especially convenient if the device 300 is a wearable device worn on the body or clothing of a person. The wireless module 340 may, for example, be any of a near field communication (NFC), wifi, short-range wireless, Bluetooth or cellular network module etc. In some examples the wireless module may be a NFC module, which is especially suitable for wearable devices, as NFC protocols can be handled by a simple processor and require relatively little electrical power.

The device 300 may also include a power module 350 for delivering power to the various components of the device. The power module may be a battery and/or may be a module configured to receive power through a power cable or a wireless connection to a power source. In some examples the wireless module 340 and power module 350 may be provided as a single module capable of both receiving power wirelessly and transmitting wireless communications from the processor to an external device. In some examples the wireless module 340 and power module 350 may take the form of a near field communication (NFC) module which is configured to receive wireless power and transmit the output data from the processor to an external device. In some examples, the device 300 is configured to receive power wirelessly and comprises a low dropout regulator (LDO) to regulate and supply wirelessly received power to the processor and the circuitry. The LDO may help to ensure a desired voltage is provided to various components of the device despite fluctuations in received wireless power.

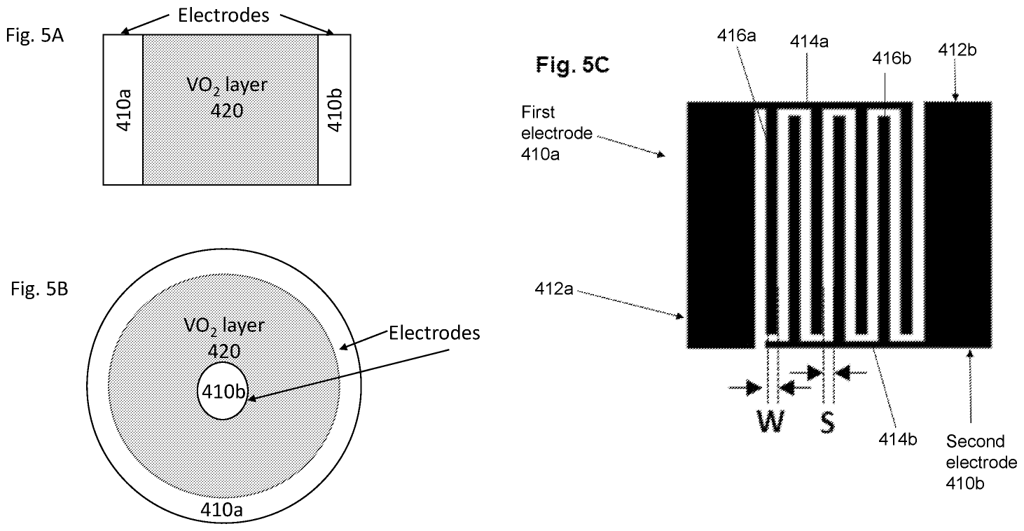

Examples of possible structures of the nicotine sensor 110, 310 are shown in FIGS. 4A, 4B and 5A to 5C.

The nicotine sensor 110, 310 may include a first electrode 410a, a second electrode 410b and a VO₂ layer between the first electrode and the second electrode. The device circuitry 120, 320 may be configured to detect a change in electrical resistance of the VO₂ layer between the first electrode and the second electrode. In this context detecting a change in electrical resistance may refer to either detecting the present electrical resistance or a parameter related thereto and thus detecting changes over time or directly detecting relative changes in the electrical resistance or a parameter related thereto. The VO₂ layer may be exposed to environment such that the VO₂ layer comes into contact with air or gases in the environment. For instance the VO₂ layer may be on a surface of the sensor.

In one example, shown in FIG. 4A, the nicotine sensor comprises a substrate 430, a VO₂ layer 420 on top of the substrate and at least two electrodes 410a, 410b on top of the VO₂ layer, such that an electrical current may pass through the VO₂ layer 420 between the electrodes 410a, 410b.

In the example of FIG. 4A, the first electrode 410a and second electrode 410b are provided on the same side of the VO₂ layer 420. In another example, shown in FIG. 4B, the first electrode 410a and second electrode 410b may be positioned on opposite sides of the VO₂ layer 420. In both cases, an electrical current may pass through the VO₂ layer 420 between the electrodes 410a, 410b.

The electrodes 410a, 410b may cover a portion of the surface of the VO₂ layer 420 on which they are mounted and leave a portion of the surface exposed to the environment. The electrodes 410a, 410b may have any suitable pattern and some possible examples are shown in FIGS. 5A to 5C, which are top down views of a surface of the electrode. In order to increase the sensitivity of the sensor to the presence of nicotine, in some examples less than half of the sensor surface or even less than twenty percent of the sensor surface may be occupied by the electrodes, with some or all of the remainder of the surface comprising a VO₂ layer exposed to the environment.

In the example of FIG. 5A, the first and second electrodes 410a, 410b are positioned proximate opposite edges of the sensor surface, which maximises the exposed area of the VO₂ layer between the electrodes. In FIG. 5B the electrode has a circular design, with the first electrode 410a being a ring and the second electrode 410b being a circle inside the ring and the VO₂ layer in between. However, other geometric shapes and configurations, such as oblongs or square rings, could be used.

In some implementations, the first electrode 410a and second electrode 410b may be interdigitated electrodes. This has the advantage that there may be a relatively large exposed area of VO₂ between the electrodes but the sensor may be sensitive to variations of electrical resistance in any one of the areas between the interdigitated fingers. FIG. 5C shows an example in which each of the first 410a and second 410b electrodes comprises a contact pad 412a, 412b for electrical contact with the device circuitry (e.g. 120, 320 in FIGS. 1 and 3), a base member 414a, 414b extending from the contact pad and a plurality of fingers 416a, 416b extending from the base member towards the base member of the other electrode. The fingers are said to be interdigitated as some of the fingers of the first electrode extend into gaps between fingers of the second electrode and vice versa. In some examples, the width of the fingers W and the gap S between adjacent fingers of opposite electrodes may be approximately the same, as this provides a sensor which is highly sensitive to resistance changes in small parts of the VO₂ layer.

The sensor may be relatively small and compact. In one example, the sensor has an area of between 8 and 15 mm². In one example the sensor comprises interdigitated electrodes, the fingers and/or contact pad have a length of about 2-4 mm and the sensor width as measured from the start of the contact pad of the first electrode to the end of the contact pad of the second electrode is 3-5 mm. In some examples the electrodes may comprise copper. The sensor may comprise hard materials such as the sensor substrate. However, despite comprising hard materials, where the sensor is small, it is easy to incorporate into a wearable device for attachment to skin or clothing, even if a main body of the wearable device comprises a flexible substrate, such as a polymer or elastomer.

As mentioned above, the nicotine sensor comprises a VO₂ layer. The VO₂ layer may be in crystalline form (e.g. a crystalline VO₂ layer). Compared to VO₂ in amorphous form, VO₂ in crystalline form is far less susceptible to dissolving in water and thus provides a much more robust sensor. Furthermore, VO₂ in amorphous form may be more susceptible to oxidation due to reaction with oxygen and/or moisture from environment which may change the phase of the VO₂ to the more thermodynamically stable phase of V₂O₅ or its hydroxides, and thus change the electrical properties of the VO₂ even in the absence of nicotine.

The nicotine sensor comprising a VO₂ layer may be fabricated using a two-step process comprising deposition of an amorphous VO₂ layer on a suitable support, and a subsequent annealing step to crystallise the amorphous VO₂ layer to a crystalline VO₂ layer, as described in Australian Patent Application No. 2017210601, which published with Publication Number AU2017210601A1 on 21 Feb. 2019, the content of which is incorporated herein by reference.

By way of example, an amorphous VO₂ layer can be deposited onto a suitable support using pulsed DC magnetron sputtering using a vanadium target. As used herein, the term “pulsed DC magnetron sputtering” refers to the process of depositing materials onto the surface of a support utilising a strong magnetic field near the support. The support for the VO₂ layer may be selected from the group consisting of glass, silicon dioxide (e.g. quartz), silicon, stainless steel, aluminium, ceramic, or reinforced plastic. In one example, the support may be a silicon dioxide (SiO₂), for example a SiO₂ wafer which may be plasma-cleaned prior to depositing the amorphous VO₂ film thereon. The VO₂ layer 420 in FIG. 4A may be a VO₂ film as described above and the substrate 430 in FIG. 4A may be a support as described above.

In an example, the deposition of an amorphous VO₂ layer on a support by pulsed DC magnetron sputtering may comprise a sputtering pressure of about 2.8×10⁻³ Torr (3.7×10⁻¹ Pa), a base pressure of about 4×10⁻⁷ Torr (5.3×10⁻⁵ Pa), may have a sputtering power of about 200 W, may have a pulse frequency of about 25 kHz, may have a reverse time of about 5 μs, may have a support temperature of about room temperature (i.e. about 20° C. to about 25° C.), may have the support placed at a distance from the vanadium target of about 12 cm, may have a sputtering gas having an O₂ partial pressure in Ar of about 30%, may have an O₂ flow rate of about 5.25 sccm and may have an Ar flow rate of about 12.25 sccm. The amorphous layer may then be annealed to crystallise the amorphous VO₂ layer into a crystalline VO₂ layer at a pressure of about 250 mTorr (33.3 Pa), a temperature of about 550° C., and for a period of time of about 90 min.

The term ‘VO₂ layer’ refers a layer comprising the presence of the vanadium (V) and oxygen (O₂), at least partially including VO₂ and preferably including at least 80% VO₂. The VO₂ may therefore also include one or more of trace impurities, dopants and concentrations of vanadium oxide in other configurations undetectable using X-ray photoelectron spectroscopy (XPS) and/or Raman spectroscopy. The term ‘crystalline VO₂ layer’ is intended to mean a layer that has a corresponding X-ray diffraction spectrum showing crystalline peaks characteristic of VO₂, where the layer forms a crystal lattice with a degree of long-range atomic order. In the fabricated crystalline VO₂ layer of the sensor, it will be appreciated that the vanadium is predominantly present in an oxidation state of +4, also referred to as vanadium (IV), or V^{4′}. It will be appreciated that the crystalline vanadium oxide layer of the sensor comprises predominantly vanadium (IV) and oxygen, in the form of vanadium (IV) oxide (VO₂).

Interdigitated (IDT) electrodes having a width and gap of 100 μm can then be fabricated on the VO₂ surface, for example after performing standard photolithography, deposition of 200 nm of Au with 20 nm Cr adhesion, followed by lift-off process. The resulting sensor can be diced to a suitable size, for example to a size of 4×4 mm².

As mentioned above, the processor 130, 330 is configured to receive, from the circuitry 120, 320, a signal representative of the detected change in the electrical resistance of the nicotine sensor 110, 310. The signal representative of the detected change in the electrical resistance of the nicotine sensor may for example be a voltage signal or a current signal. In the case of a voltage signal, the voltage signal may be based on a potential difference across the nicotine sensor or a potential difference across a resistor in series with the nicotine sensor. In some examples, the circuitry may comprises a Wheatstone bridge. A Wheatstone bridge is a type of circuit which is very sensitive to small changes in resistance.

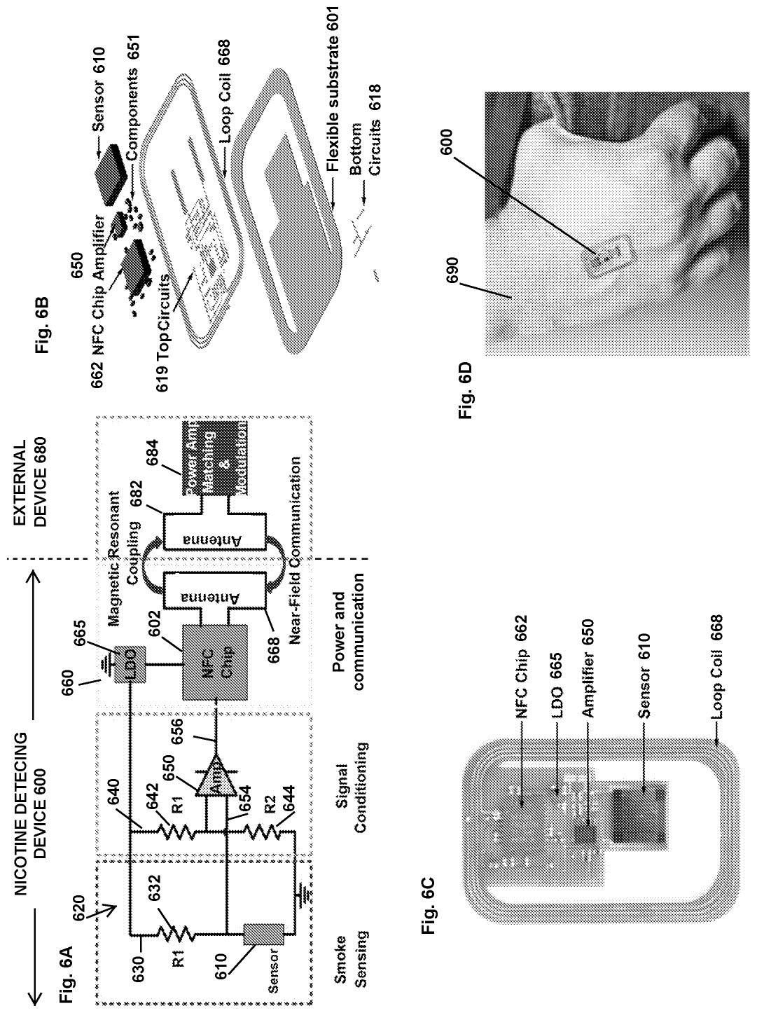

FIG. 6A shows a schematic example of one possible design of a nicotine detecting device 600. The device 600 comprises a nicotine sensor 610 which may be the same as the nicotine sensor 110, 310 described in the examples above. The nicotine sensor 610 forms part of a Wheatstone bridge 620. The Wheatstone bridge 620 comprises a first resistor 632 and nicotine sensor 610 in series on a first branch 630 of the bridge and a third resistor 642 and fourth resistor 644 in series on a second branch 640 of the bridge. The nicotine sensor 610 thus acts as a second resistor on the first branch, such that small changes in the electrical resistance of the nicotine sensor can be detected.

The first branch 630 is connected in parallel with the second branch 640 and a voltage may be applied across the first and second branches by a power supply (e.g. LDO 665). The first and third resistors 632, 642 have an electrical resistance of R1, while the fourth resistor has an electrical resistance of R2 and the nicotine sensor 610 has an electrical resistance which varies depending on the presence of nicotine. As the first branch 630 and second branch 640 of the Wheatstone bridge 620 are in parallel, the same potential difference is applied across both branches. On the first branch, as the first resistor 632 and nicotine sensor 610 are in series, the potential drop across the first resistor 632 depends upon the resistance of the nicotine sensor 610. On the second branch the voltage drop across the third resistor 642 depends on the relative resistances R1 and R2 of the third and fourth resistors 642, 644. For example, if R1 is double R2, then two thirds of the voltage drop will be across the third resistor 642 which has a resistance of R1.

The nicotine sensor 610 may be designed to have a resistance of R2, the same as the fourth resistor 644, when no nicotine is present. In that case, the voltage drop across the first resistor 632 of the first branch will be the same as the voltage drop of the third resistor 642 of the second branch. However, if nicotine is present and the resistance of the nicotine sensor drops, then the voltage drop across the first resistor 632 will be greater. In this way by monitoring the voltage drop it is possible to detect a change in the resistance of the nicotine sensor and thus detect the presence of nicotine. Wheatstone bridges may be highly sensitive to small changes in resistance so that it is possible to detect even small changes in resistance. The Wheatstone bridge is thus part of the circuitry for detecting a change in the electrical resistance of the nicotine sensor.

There are various ways in which the voltage drop or change in resistance can be monitored. In the example of FIG. 6A, the device circuitry comprises a differential amplifier 650 configured to generate an output 656 based on a difference between a first input signal 652 and a second input signal 654. The circuitry is configured such that the first input signal varies depending on the electrical resistance of the nicotine sensor 610. For example, the first input of the differential amplifier may be connected to a part of the second branch 640 between the third and fourth resistors, while the second input of the differential amplifier may be connected to a part of the first branch between the first resistor and the nicotine sensor. In this way, changes in the resistance of the nicotine sensor will change the potential difference at the second input while the potential difference at the first input remains the same, leading to a change in the output of the differential amplifier.

In some examples, the first and third resistors may have the same resistance R1 and the nicotine sensor 610 may be configured to have the same resistance R2 as the fourth resistor in the absence of nicotine; in this case when no nicotine is present the differential amplifier will have an output of zero. However, when nicotine is present the differential amplifier will output a signal proportional to the change in resistance of the nicotine sensor. In other examples the nicotine sensor may be configured to have a different resistance than the fourth resistor R2 when no nicotine is present, in such cases when no nicotine is present and a predetermined voltage from the power supply is applied across the Wheatstone bridge, the differential amplifier will output a predetermined voltage sent and when nicotine is present the output voltage will deviate from this predetermined valued. In both cases, the output 656 of the differential amplifier is thus a signal representative of detected changes in the electrical resistance of the nicotine sensor.

The Wheatstone bridge 620 and differential amplifier 650 arrangement shown in FIG. 6A are thus one example of circuitry for detecting a change in the electrical resistance of the nicotine sensor 610. They act as smoke or vape sensing and signal conditioning parts of the device. The device further comprises a power and communication module. While other types of power and communication modules are possible, in the example of FIG. 6A, the power and communication module comprises a NFC chip 662 for wirelessly receiving power from an external device and wirelessly sending and/or receiving communications to/from an external device. The NFC chip may be connected to an antenna 665 for wirelessly sending and/or receiving power and communication signals. As the wirelessly received power may vary in level or not be the appropriate level for the other components of the device, such as the Wheatstone bridge, the NFC chip 662 may be connected to a LDO to modulate and/or regulate the power and supply an appropriate power level to the other device components (e.g. a voltage to the Wheatstone). In the example of FIG. 6A the NFC chip 662 also acts as a processor similar to the processor 130, 330 described in the examples of FIGS. 1 and 3 to receive a signal from the circuitry (e.g. differential amplifier output 6556) and output data to an external device.

FIG. 6A also shows an external device 680 which is separate from the nicotine detecting device 600. The external device may for example be a smart watch, smart phone, computer or tablet computer etc. The external device comprises a power amplifier and matching and modulation circuit 684 and an antenna 682 for sending power wirelessly to the nicotine detecting device 600. The external device 680 may also have further components such as a display (not shown) and a processor (not shown) for processing data received from the nicotine detecting device 600.

The nicotine sensing device may be a wearable device. For example, the device may comprise a flexible substrate and the nicotine sensor and the processor may be mounted on the flexible substrate. In such cases, a first side of the flexible substrate may be configured for attachment to skin and the nicotine sensor may be mounted to a second side of the flexible substrate which is opposite to the first side. In this way the nicotine sensor may be exposed to the air and able to come into contact with ambient gases, such as smoke or vape. This is in contrast to sweat sensors, in which the sensor is positioned on the side of the flexible substrate facing the skin.

FIG. 6B is a diagram showing an exploded view of the various layers of an exemplary wearable nicotine detecting device 600. FIG. 6C shows a top down view of the device and FIG. 6D shows a view of the device when worn on a person's skin. The same reference numerals are used for like parts as in FIG. 6A.

As shown in FIG. 6B, the device 600 comprises a plurality of components mounted on a flexible substrate 601. The flexible substrate may be any suitable elastomer, such as but not limited to polyamide and may be configured for attachment to a user's skin, for example by adhesion. In one example the thickness of the flexible substrate is 75 μm. A conductive layer is mounted on the flexible substrate. The conductive layer may comprise various conductive lines 619 for connecting components of the device and a loop coil antenna 668 for sending and receiving wireless signals. There may also be some conductive lines 618 on the bottom skin facing side of the flexible substrate, which may connect to circuits on the top side through one or more vias. In one example the conductive lines may comprise copper and may have a thickness of 18 μm.

As shown in FIG. 6B, the flexible substrate may include some gaps or apertures to enhance breathability and flexibility. A electronic component layer may be formed by electronic components mounted on the flexible substrate 601. The electronic components may include the NFC chip 662, nicotine sensor 610, amplifier 650 and other components 651 (such as resistors of the Wheatstone bridge). The various electronic components and connected by the conductive lines 619, 618. As shown in FIG. 6C, the loop coil antenna 668 may run around the circumference of the device so as to provide a compact structure.

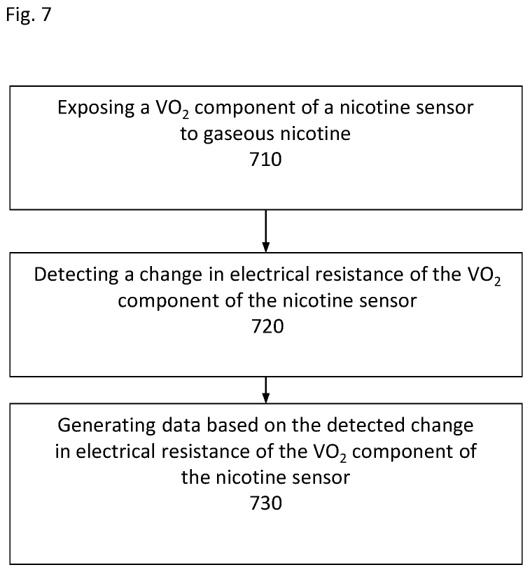

FIG. 7 shows an example method 700 of detecting gaseous nicotine. At block 710 the method comprises exposing a vanadium oxide (VO₂) component of a nicotine sensor to gaseous nicotine. Then at block 720 the method further comprises detecting a change in an electrical resistance of the VO₂ component of the nicotine sensor. Then at block 730 the method further comprises generating data based on the detected change in electrical resistance of the VO₂ component of the nicotine sensor.

The method may use any of the nicotine detecting devices described in the examples above. At block 720, detecting the change in resistance of the VO₂ component of the nicotine sensor may comprises detecting a change in a current or voltage signal generated by circuitry connected to the nicotine sensor.

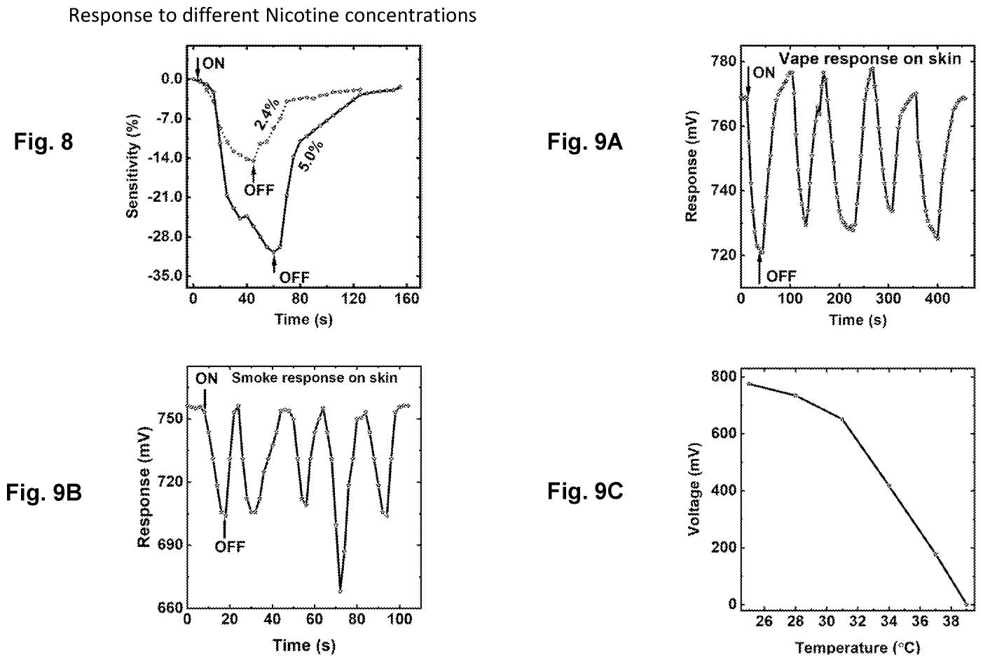

FIG. 8 shows the results of a test of the sensitivity of a nicotine detecting against different concentrations of nicotine. A device of the type described above was placed inside a closed chamber and exposed to nicotine having concentration of 2.4% and 5.0%. The sensitivity of the device was then calculated in accordance with the following equation:

S=(Va−Vg)/Va×100Equation 2

Where S is the sensitivity of the device, Va is the potential difference across the nicotine sensor when exposed to air and Vg is the potential difference across the nicotine sensor when exposed to nicotine.

More specifically, FIG. 8 shows sensitivity against time for a device of the type described above when exposed to vape having 2.4% concentration of synthetic nicotine and when exposed to vape having 5.0% concentration of synthetic nicotine from a commercial device. The arrow ON indicates the point in time at which the vape was introduced to the chamber and the OFF arrow indicates the point in time at which the introduction of vape was stopped. It can be seen that the sensitivity to 5.0% nicotine is almost double the sensitivity to 2.4% nicotine and so the response of the device is approximately linear to variations in concentration. Therefore it may be possible to monitor not only the presence of nicotine, but also the level of exposure.

FIG. 9A shows the response of the device to consecutive exposures to vape. The y axis shows the output response of the device in mV output and the x axis shows variation over time. The device was attached to the skin of a person and repeatedly exposed to puffs of vape having 5% vaporized synthetic nicotine. The response data was logged by an external device which wirelessly received the data from the processor (e.g. NFC chip) based on the signal from the differential amplifier and Wheatstone bridge.

In FIG. 9A, the arrow ON indicates the introduction of first batch of vape and the OFF arrow indicates when the first batch stopped. The following peaks and troughs correspond to the further batches of vape. It can be seen that despite repeated exposures over a short period of time, the device kept a good response and recovered quickly after each exposure. FIG. 9B is similar to FIG. 9A, but shows data gathered using puffs of cigarette smoke instead of vape. This demonstrates the device can be used to detect both vape and cigarette smoke.

In some examples, the nicotine detecting method of FIG. 7 may be carried out between temperatures of 10 and 50 degrees Celsius. VO₂ undergoes phase changes around 70 degrees Celsius and these alter the electrical properties of the VO₂, so the above range gives a safety margin. In other examples, the method may be carried out between 15 and 35 degrees Celsius which corresponds to a broad range of room temperatures. It is to be noted that, while many other gas sensors require high temperatures to operate, the present application provides a device which, in at least some implementations, is capable of monitoring for nicotine in real time at room temperature.

Variation in temperature may change the electrical resistance of the nicotine sensor. FIG. 9C is a graph showing an example of response of a device in mV against temperature. It can be seen that the response reduces as the temperature rises. However, this need not interfere with the nicotine detecting ability of the device. In normal use cases room temperature will change only very slowly, while the change in VO₂ resistance and thus response of the device in the presence of nicotine occurs very quickly, in the order of 10s of milliseconds as shown in FIGS. 8, 9A and 9B.

Accordingly, in one example the device and/or method may determine that nicotine is present in response to detecting a rapid change in response. For example, a determination that nicotine is present may be made based on detecting a change in electrical resistance of the VO₂ component of at least a predetermined amount within a predetermined period of time. The change in electrical resistance may be detected based on the data generated at block 730 of FIG. 7 (e.g. data generated by the device processor). The predetermined period of time may, for example, be a period of 1 second or less, for example a period of 50 ms. The predetermined amount may for instance be a change of at least 5% in the predetermined period of time. The data may be filtered or smoothed with a rolling average of periodic samples or otherwise so as to avoid noise, outliers or spikes in the data triggering an alert. Another approach is to select resistors for the Wheatstone bridge which have a same variance of resistance with temperature as the nicotine sensor over the operating temperature range of the device; in which case the output of the Wheatstone bridge to the amplifier will not vary with temperature.

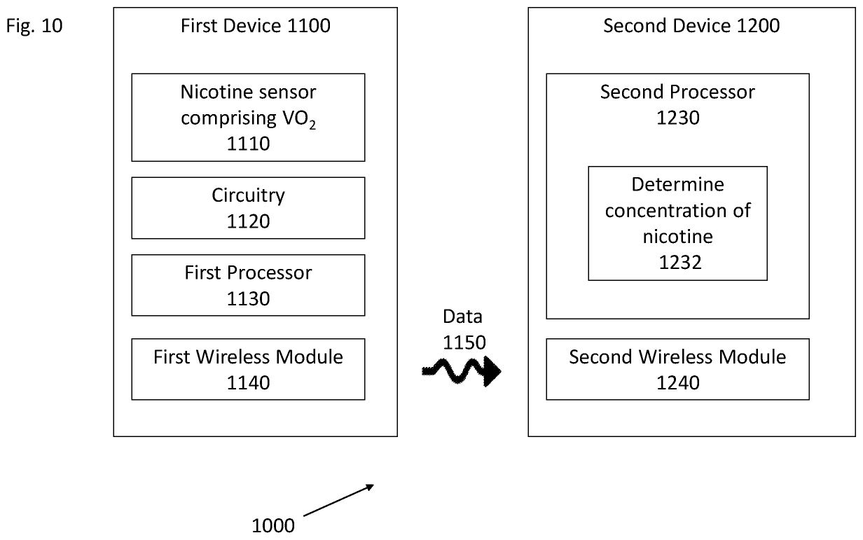

FIG. 10 shows an example system 1000 for detecting nicotine. The system comprises a first device 1100, which may be a nicotine detecting device, and a second device 1200 which may receive data wirelessly from the first device.

For example, the first device may be a wearable nicotine detecting device, such as shown in FIGS. 6A to 6D and the second device may be a smart watch, smart phone or other computing device or service. In some examples the second device may comprise a processor configured to run an app or software to provide functionality described below. For instance the second device may include a processor and machine readable storage medium storing machine readable instructions which are executable to run the app and perform any of the methods and functionality described herein.

The first device 1100 includes a nicotine sensor 1110 comprising vanadium oxide (VO₂), a first processor 1130 and circuitry 1120. The circuitry 1120 is configured to monitor an electrical property of the nicotine sensor 1110 and send a signal based on the monitored electrical property to the first processor 1130. The first processor 1130 is configured to control a first wireless communication module 1140 to send data 1150 based on the signal to the second device.

The second device 1200 comprises a second wireless communication module 1240 to wirelessly receive the data 1150 from the first device 1100 and a second processor 1230 to determine 1232 a concentration of nicotine at the nicotine sensor 1110 of the first device based on the data 1150 received from the first device.

The electrical property monitored by the circuitry 1120 may for example be an electrical resistance of the nicotine sensor and the signal based on the monitored electrical property may be a current signal or a voltage signal. The first device 1100 may have any of the features of the nicotine detecting devices described in the examples above and may be configured to perform any of the methods described in the examples above including those described in relation to FIGS. 1 to 9.

The second processor 1200 may be configured to perform one or more of the following operations. In one example the second processor is configured to determine a concentration of nicotine at the nicotine sensor by using calibration data based on a relationship between nicotine concentration and resistance of VO₂ when exposed to nicotine. This makes it possible to design a simple, low power first device and offload most of the processing to the second device, which may have a steady source of power and a more powerful processor. This is especially useful if the first device is a disposable wearable which is to be abandoned after use. In one example, the second processor is configured to display information relating to the nicotine concentration on a display of the second device. In this way the information can be made available to the user even if the first device does not have a display. In one example, the second processor is configured to output an alert in response to the nicotine concentration matching alert criteria. The alert criteria may be specified by clinicians or the designer of the first device. In some examples the alert criteria may include the nicotine concentration, or values in the output data 1150, passing a predetermined threshold or changing by at least a predetermined amount (e.g. 5%) in a predetermined period of time (e.g. 50 ms). In one example, the second processor is configured to send the determined nicotine concentration for storage on a storage medium, such as a local storage of the second device and/or a cloud storage.

It will be appreciated that there are many different use cases for the device method and systems described in this application. The processor the nicotine detecting device 130, 330, 662 1130 is configured to output data based on a detected change in an electrical property of the nicotine sensor 110, 310, 610, 1110. The content of the data will depend upon the design of the nicotine detecting device and extent to which processing is carried out on the nicotine detecting device itself or an external device.

In some examples the output data may comprise mV response values. For instance voltage values generated by the device circuitry, such as through an amplifier and circuitry monitoring the resistance of the nicotine sensor. This is particularly appropriate if the nicotine detecting device is a simple lower power device, such as a wearable comprising a NFC module. In such cases, most of the processing may be carried out on an external device which receives the output data. In some other examples the nicotine detecting device may have a more powerful processor, such that more processing can be carried out on the device and the output data may comprise a calculated nicotine concentration; for instance this approach may be used if the device is a stationary nicotine detector to be mounted to a wall or ceiling of an indoor setting.

In still other examples, the output data may be an alert indicating presence of nicotine. This approach may be used in the case of a stationary detector, but may also prove useful where the device is a wearable and the provider of the device wishes to monitor the user to determine if they are smoking or vaping. For example, some insurance companies may make lower premiums conditional on not smoking or vaping and wearing the device for a period of time. In one example, the nicotine detecting device may be in the form of a wearable and may include cellular communication module and/or a SIM card and the processor may be configured to send an alert to the insurer or device provider if nicotine, or a smoking or vaping event is detected. The processor may for instance be configured to determine that nicotine is present or detect a smoking or vaping event based on the signal from the circuitry meeting predetermined criteria.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A device for detecting nicotine comprising:
a nicotine sensor comprising vanadium oxide (VO₂), the nicotine sensor having an electrical resistance which is sensitive to presence of nicotine;
circuitry configured to detect a change in the electrical resistance of the nicotine sensor;
and a processor configured to receive, from the circuitry, a signal representative of the detected change in the electrical resistance of the nicotine sensor and configured to output data based on the detected change in electrical resistance of the nicotine sensor.

2. The device of claim 1 wherein the nicotine sensor includes a first electrode, a second electrode, a VO₂ layer between the first electrode and the second electrode, and wherein the circuitry is configured to detect a change in electrical resistance of the VO₂ layer between the first electrode and the second electrode.

3. The device of claim 2 wherein the first electrode and second electrode are interdigitated electrodes.

4. The device of claim 1 wherein the signal representative of the detected change in the electrical resistance of the nicotine sensor is a voltage signal.

5. The device of claim 4 wherein the voltage signal is based on a potential difference across the nicotine sensor or a potential difference across a resistor in series with the nicotine sensor.

6. The device of claim 4 wherein the circuitry comprises a Wheatstone bridge.

7. The device of claim 1 wherein the circuitry comprises a differential amplifier configured to generate an output based on a difference between a first input signal and a second input signal, wherein the circuitry is configured such that the first input signal varies depending on the electrical resistance of the nicotine sensor.

8. The device of claim 1 wherein the device is a wearable device comprising a flexible substrate and wherein the nicotine sensor and the processor are mounted on the flexible substrate.

9. The device of claim 8 wherein a first side of the flexible substrate is configured for attachment to skin and the nicotine sensor is mounted to a second side of the flexible substrate which is opposite to the first side.

10. The device of claim 1 further comprising a wireless module for communicating the output data to an external device.

11. The device of claim 1 wherein the device is configured to receive power wirelessly and comprises a low dropout regulator (LDO) to regulate and supply wirelessly received power to the processor and the circuitry.

12. The device of claim 10 wherein the wireless module is a near field communication (NFC) module which is configured to receive wireless power and transmit the output data from the processor to an external device.

13. The device of claim 1 wherein the nicotine sensor comprises VO₂ in crystalline form.

14. The device of claim 1 wherein the device is configured to detect changes in nicotine concentration in real time by detecting changes in electrical resistance of the nicotine sensor in real time.

15. A method of detecting gaseous nicotine, the method comprising:
exposing a vanadium oxide (VO₂) component of a nicotine sensor to gaseous nicotine;
detecting a change in an electrical resistance of the VO₂ component of the nicotine sensor;
generating data based on the detected change in electrical resistance of the VO₂ component of the nicotine sensor.

16. The method of claim 15 wherein the method is carried out between temperatures of 15 to 50 degrees Celsius.

17. The method of claim 15 wherein detecting the change in resistance of the VO₂ component of the nicotine sensor comprises detecting a change in a current or voltage signal generated by circuitry connected to the nicotine sensor.

18. The method of claim 15 comprising determining that nicotine is present in response to detecting, based on the generated data, a change in electrical resistance of the VO₂ component of at least a predetermined amount within a predetermined period of time.

19. A system for detecting nicotine comprising a first device and a second device, wherein:
the first device includes a nicotine sensor comprising vanadium oxide (VO₂), a first processor, circuitry configured to monitor an electrical property of the nicotine sensor and send a signal based on the monitored electrical property to the first processor, and the first processor is configured to control a first wireless communication module to send data based on the signal to the second device; and
the second device comprises a second wireless communication module to wirelessly receive the data from the first device and a second processor to determine a concentration of nicotine at the nicotine sensor of the first device based on the data received from the first device.

20. The system of claim 19 wherein the second processor is configured to perform at least one of the following operations:
determine a concentration of nicotine at the nicotine sensor by using calibration data based on a relationship between nicotine concentration and resistance of VO₂ when exposed to nicotine;
display information relating to the nicotine concentration on a display of the second device;
output an alert in response to the nicotine concentration matching alert criteria; and/or
send the determined nicotine concentration for storage on a storage medium.
